SEQUENCE LISTING (9 sequences)

SEQ ID NO: 1 - DNA, 1029 bp, Candida antarctica
  CDS (1)..(1026)
  sig_peptide (1)..(75)
  mat_peptide (76)..(1026)
atgaagctactctctctgaccggtgtggctggtgtgcttgcgacttgcgttgcagccactcctttggtgaagcgtctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctcaacgcagttgggttacacaccctgctggatctcacccccgccgttcatgctcaacgacacccaggtcaacacggagtacatggtcaacgccatcaccgcgctctacgctggttcgggcaacaacaagcttcccgtgcttacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcaccaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggccgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcagctgcagccatcgtggcgggtccaaagcagaactgcgagcccgacctcatgccctacgcccgcccctttgcagtaggcaaaaggacctgctccggcatcgtcaccccctga

SEQ ID NO: 2 - protein, 342 aa, Candida antarctica
MKLLSLTGVAGVLATCVAATPLVKRLPSGSDPAFSQPKSVLDAGLTCQGASPSSVSKPILLVPGTGTTGPQSFDSNWIPLSTQLGYTPCWISPPPFMLNDTQVNTEYMVNAITALYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 3 - protein, 319 aa, Artificial sequence
  Hyphozyma species
FTPFPTGADPAFTQSQATLDAGLTCQSGSPSSQKNPILLVPGTGNTGPQSFDSNWIPLSAQLGYSPCWVSPPPFMLNDSQINAEYIVNAIHTLSSGSGSKVPVLTWSQGGLAAQWALTFFPSTRNKVDRLMAFAPDYKGTVEAGLLDAFGLSAPSVWQQTAQSAFVTALDQAGGLNQIVPTTNLYSATDEVVQPQFANGPPDSSYLSNGKNIQAQSICGPLFIIGHAGSLYSQFSYVVGKSALASPTGQAQSSDYSIKDCNPAPANPLTAQQKLDSAAIILVAGKNIVTGPKQNCEPDLMPYARKYRIGKKTCSGVITG

SEQ ID NO: 4 - protein, 336 aa, Ustilago maydis
MKTTSVISALVTLASIIRAAPLASSDPAFSTPKATLDAGLECQTGSPSSQTKPILLVPGTGANGTQTFDSSWIPLSAKLGFSPCWISPPPFMLNDSQVNVEYIVNAVQTLYAGSGSKKVPVLTWSQGGLATQWALTFFPSIRNQVDRLMAFAPDYKGTIEAGLLSTFGLASQSVWQQQAGSAFVTALKNAGGLTSFVPTTNLYSFFDEIVQPQVFNSDADSSYLGNSKNIQAQTVCGGFFVIDHAGSLTSQFSYVVGKSALTSSSGVANSADYSSKDCKASPADDLSAKQKADASALLFVAAGNLLAGPKQNCEPDLKPYARQFAVGKKTCSGTIN

SEQ ID NO: 5 - protein, 445 aa, Gibberella zeae
APSYSDLESRQLIGGLLKGVDGTLETVVGGLLGTLRKAIDSGDRDKTLDILHVLEPAKKHKNVEEAFAALEKISKSKPKTIIDYSAQLIVNGLISGNTLDLFAYAKGLVSAQNGSNNKNRNPPKEVYPKVANCDASYTTSEAKLRAAIHIPPTFTYGEKPPVILFPGTGSTGFTTYRGNFIPLLTDVEWADPVWVNVPVLLLEDAQVNAEYAAYALNYIASLTKRNVSVIAWSQGNIDVQWALKYWPSTRKVTTDHVAISADYKGTILANIGGATGLINTPAVVQQEAGSTFINTLRSNDGDSGYIPTTSLYSSLFDEVVQPQEGAGASAYLLDARDVGVTNAEVQKVCTGKLGGSFYTHESMLANPLTFALAKDALTHEGPGTISRLDLADVCNRSLAPGLGLKDLLITENAVVIAALSLVLYLPKQIDEPAIKQYALEATGTC

SEQ ID NO: 6 - protein, 455 aa, Debaryomyces hansenii
HPTKELERRDLISNIDDIVNSTIDNGEAHKDNAKSAITDIFDKINDGIKQDIDNLKEVGKSIADLIKSVVPTEDLSTPEGVQAYLGQLFENGEDLFKNSIDMVGHGLKPGSIAGNFEGFSDEINTSDNFNVKEPEGSVYPQAESEDPSFSLSEEQLRSAIQIPEEFQYGNGSKSPVILVPGTGSKGGMTYASNYAKLLKETDFADVVWLNVPGYLLDDAQNNAEYVAYAINYISGISNNKNVSIISWSQGGLDTQWALKYWASTRSKVSDFIPISPDFKGTRMVPVLCPSFPKLSCPPSVLQQEYNSTFIETLRADGGDSAYVPTTSIYSGFDEIVQPQSGKGASGLINDNRNVGVTNNEVQTICPDRPAGKYYTHEGVLYNPVGYALAVDALTHEGPGQLSRIDLDTECGRIVPDGLTYTDLLATEALIPEALVLILSYDDKTRDEPEIRSYAQ

SEQ ID NO: 7 - protein, 440 aa, Aspergillus fumigatus
AVIPRGAVPVASDLSLVSILSSAANDSSIESEARSIASLIASEIVSKIGKTEFSRSTKDAKSVQEAFDKIQSIFADGTPDFLKMTREILTVGLIPADIVSFLNGYLNLDLNSIHNRNPSPKGQAIYPVKAPGDARYSVAENALRAAIHIPASFGYGKNGKKPVILVPGTATPAGTTYYFNFGKLGSAADADVVWLNIPQASLNDVQINSEYVAYAINYISAISESNVAVLSWSQGGLDTQWALKYWPSTRKVVDDFIAISPDFHGTVMRSLVCPWLAALACTPSLWQQGWNTEFIRTLRGGGGDSAYVPTTTIYSTFDEIVQPMSGSQASAILSDSRAVGVSNNHLQTICGGKPAGGVYTHEGVLYNPLAWALAVDALSHDGPGDPSRLDLDVVCGRVLPPQLGLDDLLGTEGLLLIALAEVLAYKPKTFGEPAIASYAH

SEQ ID NO: 8 - protein, 401 aa, Aspergillus oryzae
LPSSSETVEANCVKPYLCCGELKTPLDSTLDPILLDLGIDAASIVGSVGLLCLIPSKALTCLNGYAIIDLNSIHRHNPSPENLSIYPYKAKSDAPYSIAENTLRAAIHIPRSFSHKRDKKIPVLLVPGTAVPAAITFYFNFGKLRRALPESELVWIDLPQASLDDIQLSAEYVAYALNYVSALTSSKIAVISWSQGALDIQWALKYWPSTRSVVNDFIAISPDFHGTIVKWLVCPLLNDLACTPSIWQQGWDANFIQALRSQGGDSAYVTTTTIYSSFDKIVRPMSGENASARLLDYRGVGVSNNHLQTICANNAAGGLYTHEGVLYNPLAWALTVDALLHDGPSNITRIDTQKICEQVLPPYLELTDMLGTEALLLVALAKILTYSPKVSGEPDIAKYAY

SEQ ID NO: 9 - protein, 388 aa, Neurospora crassa
LPTTSEPVHHESVRAIGELSHRDELHDAGVVWNKVVRQSPLVAPTDPRDSFNNQNPDVPGVGYPRSSDADPAFTIPEAKLRSAIYLPSGFNSSTNRQVVLFVPGTGAYGHESFADNLLKVITNAGAADAVWVNVPNAMLDDVQSNAEYIAYAISYVKALIGDDRDLNVIGWSQGNLATQWVLTYWPSTAPKVRQLISVSPDFHGTMLAYGLCAGNFGKVAKAGAPCPPSVLQQLYSSNLINTLRAAGGGDAQVPTTSFWSRLTDEVVQPQAGLTASARMGDARNKGVTNVEVQTVCGLSVGGGQYGHSTLMAHPLVAAMTLDALKNGGPASLSRIRSQMFRACSNVVAPGLQLTDRAKTEGLLATAGARMGAFPTKLLREPALRQYAA

(19) United States
(12) Patent Application Publication
(10) Pub. No.: US 2013/0023028 A1
(43) Pub. Date: Jan. 24, 2013

(54) Variants Of A Polypeptide With Lipolytic Activity and Improved Stability

(71) Applicants: Allan Svendsen, Horsholm (DK); Sangeeta Naik, Bangalore (IN); Saikia Rakhi, Bangalore (IN); Aditya Basu, Bangalore (IN); Pritish Paul, Bangalore (IN); Santhosh Maypadum Vasu, Bangalore (IN); Leonardo De Maria, Frederiksberg (DK); Michael Skjot, Roskilde (DK)

(72) Inventors: Allan Svendsen, Horsholm (DK); Sangeeta Naik, Bangalore (IN); Saikia Rakhi, Bangalore (IN); Aditya Basu, Bangalore (IN); Pritish Paul, Bangalore (IN); Santhosh Maypadum Vasu, Bangalore (IN); Leonardo De Maria, Frederiksberg (DK); Michael Skjot, Roskilde (DK)

(73) Assignees: Novozymes South Asia Pvt. Ltd., Bangalore (IN); Novozymes A/S, Bagsvaerd (DK)

(21) Appl. No.: 13/511,919

(22) PCT Filed: Dec. 2, 2010
(86) PCT No.: PCT/EP2010/068764

§ 371 Date: Sep. 26, 2012

(30) Foreign Application Priority Data
Dec. 3, 2009 (IN) 2986/CHE/2009

(51) Int. Cl. C12N 9/20; C12N 15/63; C12N 1/19; C12N 5/10; C12N 1/15; C12N 15/55; C12N 1/21

(52) U.S. Cl. 435/198; 536/23.2; 435/320.1; 435/252.3; 435/252.31; 435/252.35; 435/252.33; 435/252.34; 435/325; 435/348; 435/419; 435/254.11; 435/254.2; 435/254.22; 435/254.23; 435/254.21; 435/254.3; 435/254.7; 435/254.4; 435/254.8; 435/254.5; 435/254.6

(57) ABSTRACT

The invention relates to a method of preparing a variant of a parent polypeptide comprising: (a) providing an amino acid sequence of a parent polypeptide; (b) substituting at least one amino acid residue at a position in the sequence corresponding to any of positions: 41, 83, 129, 207 or 284 in SEQ ID No: 2; (c) selecting a variant with lipolytic activity, which compared to the parent polypeptide has improved stability, and has an amino acid sequence with at least 60% identity to the mature polypeptide of SEQ ID No: 2; and (d) recovering the variant.

### REFERENCE TO A SEQUENCE LISTING

This application contains a Sequence Listing in computer readable form. The computer readable form is incorporated herein by reference.

### BACKGROUND OF THE INVENTION

Candida Antarctica lipase B (CALB) has been found useful in catalyzing a great number of reactions such as interesterification, ester hydrolysis and ester synthesis including both region- and enantio-selective synthesis as well as reactions such as the reaction of peroxycarbolic acids. Like other lipases CALB has the same mechanism of action as a serin protease with an active site triade consisting of Ser105, Asp187 and His224. CALB has been described in WO88/02775 and by Uppenberg et al. 1994 Structure 2:293-308. The latter describing the amino acid sequence and three-dimensional (3D) structure of CALB. The 3D structure can be found in the Research Collaboratory for Structural Bioinformatics Protein Data Bank (RCBS PDB) (http://www.rcsb.org/), its identifier being 1TCA.

CALB is one of the most widely used industrial enzymes (Anderson, E M, et. al. 1998 Biocatal. Biotransform. 16(3):181-204). Two lipases have been purified from Candida antarctica (Patkar, S A., et al. 1993 Ind. J. Chem. 32:76-80). The other counterpart of CALB, CALA is thermostable, calcium dependent, shows preference for sn-2 reactions and has a penchant for large ester. CALB however is not thermostable, calcium independent and prefers simple esters to larger ones. Although CALB is not thermostable it has incited more industrial interest for ester synthesis due to its substrate specificity. However, one of the biggest bottlenecks has been its fragile nature in presence of higher temperature (Rogalska, E, et al. 1993 Chirality 5 (1):24-30). Considerable thermostability of the molecule has been obtained by replacing the threonine at position 103 with glycine. Further improvements in its ability to withstand higher temperatures would enable the versatile CALB to carry out reactions at higher temperatures (Patkar, S A, et al. 1997 J. MOL. CATAL. B ENZYM. 3(1-4):51-54).

CALB can catalyse the formation of peracids from parent carboxylic acids and hydrogen peroxides. This peracid can be used for in situ epoxidation of alkenes or penicillin (Bjorkling, F., et al. 1992Tetrahedron 48(22):4587-4592). Although this would be an interesting application for CALB it is limited due to the presence of amino acids prone to oxidation close to the active site. One such amino acid is methionine 72 which has been altered to render oxidation stability to CALB (Patkar, S A, et al. 1998 Chem. Phys. Lipids 93(1-2):95-101). Mutations which would render CALB more oxidation hardy would enable the enzymes to carry epoxidations.

In non-aqueous reactions for esterification with methanol, CALB is not a stable molecule and is inactivated in a very short time (Shimada, Y., et al. 1999 JAOCS J Am Oil Chem Soc 76(7):789-793). Esterification is an important reaction for the formation of biodiesel and CALB variants which would be stable for long period of time in the presence of methanol would be very useful.

The use of CALB for catalyzing reactions in many different applications makes the identification of the various factors contributing to the stability of CALB of great interest. The versatile CALB could be made even more versatile and industrially more useful by making it thermostable, methanol stable and oxidation stable. Accordingly, it would be desirable in the art to improve the stability of polypeptides like CALB having lipolytic activity, as well as improve the specific activity of such polypeptides.

### FIELD OF THE INVENTION

The present invention relates to variants of a polypeptide having lipolytic activity wherein the variants have improved properties, such as improved stability in the presence of elevated temperatures, oxidizing conditions and/or alcohol, improved specific activity, and/or improved transesterification activity, polynucleotides encoding the variants, methods of producing the variants, and methods of using the variants.

### SUMMARY OF THE INVENTION

In a first aspect the invention relates to a method of preparing a variant of a parent polypeptide comprising: (a) providing an amino acid sequence of a parent polypeptide; (b) substituting at least one amino acid residue at a position in the sequence corresponding to any of positions: 41, 83, 129, 207 or 284 in SEQ ID No: 2; (c) selecting a variant with lipolytic activity, which compared to the parent polypeptide has an improved property, and has an amino acid sequence with at least 60% identity to the mature polypeptide of SEQ ID No: 2; and (d) recovering the variant.

In a second aspect the invention relates to an isolated variant of a parent polypeptide, wherein said variant is: (a) a polypeptide comprising a substitution at at least one amino acid residue at a position corresponding to any of positions: 41, 83, 129, 207 or 284 of SEQ ID No: 2, wherein said variant has lipolytic activity, which variant compared to the parent polypeptide has an improved property, and has an amino acid sequence with at least 60% identity to the mature polypeptide of SEQ ID No: 2; (b) a polypeptide encoded by a polynucleotide that hybridizes under at least low stringency conditions with (i) the mature polypeptide coding sequence of SEQ ID No: 1, (ii) the genomic DNA sequence comprising the mature polypeptide coding sequence of SEQ ID No: 1, or (iii) a full-length complementary strand of (i) or (ii); or (c) a polypeptide encoded by a polynucleotide comprising a nucleotide sequence having at least 60% identity with the mature polypeptide coding sequence of SEQ ID No: 1.

In a third aspect the invention relates to an isolated polynucleotide encoding the variant of the invention.

In a fourth aspect the invention relates to a nucleic acid construct comprising the isolated polynucleotide of the invention.

In a fifth aspect the invention relates to an expression vector and/or a host cell comprising the nucleic acid construct of the invention.

In a sixth aspect the invention relates to a composition comprising the variant of the invention.

### BRIEF DESCRIPTION OF THE FIGURE

FIG. 1 shows an alignment of lipase amino acid sequences.

### DETAILED DESCRIPTION OF THE INVENTION

In a first aspect the invention relates to a method of preparing a variant of a parent polypeptide comprising: (a) providing an amino acid sequence of a parent polypeptide; (b) substituting at least one amino acid residue at a position in the sequence corresponding to any of positions: 41, 83, 129, 207 or 284 in SEQ ID No: 2; (c) selecting a variant with lipolytic activity, which compared to the parent polypeptide has an improved property, and has an amino acid sequence with at least 60% identity to the mature polypeptide of SEQ ID No: 2; and (d) recovering the variant.

In some aspects the invention relates to a method of preparing a variant of a parent polypeptide, wherein the parent polypeptide consists or comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to a lipase selected from the group consisting of: Candida Antarctica lipase B (SEQ ID No: 2), Hyphozyma sp. lipase (SEQ ID No: 3), Ustilago maydis lipase (SEQ ID No: 4), Giberella zeae lipase (Fusarium graminearum lipase, SEQ ID No: 5), Debaryomyces hansenii lipase (SEQ ID No: 6), Aspergillus fumigates lipase (SEQ ID No: 7), Aspergillus oryzae lipase (SEQ ID No: 8), and Neurospora crassa lipase (SEQ ID No: 9).

In some aspects the invention relates to a method of preparing a variant of a parent polypeptide, wherein at least one further position in the amino acid sequence of the parent polypeptide corresponding to any of positions 103, 197, 223 or 278 in SEQ ID No: 2 is substituted.

In some aspects the invention relates to a method of preparing a variant of a parent polypeptide, wherein the substitutions of positions: 41, 83, 103, 129, 197, 207 or 223 of SEQ ID No: 2 are 41A, 83L, 103G, 129L, 197G, 207A, 223G or 278A.

In some aspects the invention relates to a method of preparing a variant of a parent polypeptide, wherein the variant has an improved property, such as improved stability in the presence of elevated temperatures, oxidizing conditions, alcohol, or any combination thereof.

In some aspects the invention relates to a method of preparing a variant of a parent polypeptide, wherein the variant has an improved property, such as improved specific activity and/or improved transesterification activity.

In some aspects the invention relates to a method of preparing a variant of a parent polypeptide, wherein the variant has an amino acid sequence with at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID No: 2.

### DEFINITIONS

### Variant:

The term “variant” is defined herein as a polypeptide having lipolytic activity comprising an alteration, such as a substitution, insertion, and/or deletion, of one or more (several) amino acid residues at one or more (several) specific positions of the mature polypeptide of SEQ ID No: 2. The altered polynucleotide can be obtained through human intervention by modification of the mature polypeptide coding sequence disclosed in SEQ ID No: 1; or a homologous sequence thereof. Alternatively, the variant and polynucleotide thereof can be obtained from nature.

### Wild-Type Polypeptide:

The term “wild-type polypeptide” denotes a polypeptide expressed by a naturally occurring microorganism, such as bacteria, yeast, or filamentous fungus found in nature.

### Parent Polypeptide:

The term “parent polypeptide” as used herein means a polypeptide to which a modification, e.g., substitution(s), insertion(s), deletion(s), and/or truncation(s), is made to produce the variants of the present invention. This term also refers to the polypeptide with which a variant is compared and aligned. The parent may be a naturally occurring (wild-type) polypeptide or a variant. For instance, the parent polypeptide may be a variant of a naturally occurring polypeptide which has been modified or altered in the amino acid sequence. A parent may also be an allelic variant, which is a polypeptide encoded by any of two or more alternative forms of a gene occupying the same chromosomal locus.

### Isolated Variant:

The term “isolated variant” as used herein refers to a variant isolated from a source. In one aspect, the variant is at least 1% pure, at least 5% pure, at least 10% pure, at least 20% pure, at least 40% pure, at least 60% pure, at least 80% pure, or at least 90% pure, as determined by SDS-PAGE.

### Substantially Pure Variant:

The term “substantially pure variant” denotes herein a polypeptide preparation that contains at most 10%, at most 8%, at most 6%, at most 5%, at most 4%, at most 3%, at most 2%, most 1%, or at most 0.5% by weight of other polypeptide material with which it is natively or recombinantly associated. The variants of the present invention are preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods. It is, therefore, preferred that the substantially pure variant is at least 90% pure, at least 92% pure, at least 94% pure, at least 95% pure, at least 96% pure, at least 97% pure, at least 98% pure, at least 99% pure, at least 99.5% pure, or 100% pure by weight of the total polypeptide material present in the preparation. The variants and polypeptides of the present invention are preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods.

### Mature Polypeptide:

The term “mature polypeptide” is defined herein as a polypeptide in its final form following translation and any post-translational modifications, such as N-terminal processing, C-terminal truncation, glycosylation, phosphorylation, etc. In one aspect, the mature polypeptide is amino acids 1 to 317 of SEQ ID No: 2 based on the SignalP program (Nielsen et al., 1997, Protein Engineering 10:1-6) that predicts amino acids −25 to −1 of SEQ ID No: 2 are a signal peptide.

### Mature Polypeptide Coding Sequence:

The term “mature polypeptide coding sequence” is defined herein as a nucleotide sequence that encodes a mature polypeptide having lipolytic activity. In one aspect, the mature polypeptide coding sequence is nucleotides 76 to 1026 of SEQ ID No: 1 based on the SignalP program (Nielsen et al., 1997, supra) that predicts nucleotides 1 to 76 of SEQ ID No: 1 encode a signal peptide.

### Identity:

The relatedness between two amino acid sequences or between two nucleotide sequences is described by the parameter “identity”. For purposes of the present invention, the degree of identity between two amino acid sequences is determined using the Needleman-Wunsch algorithm (Needleman & Wunsch 1970 J. Mol. Biol. 48:443-453) as implemented in the Needle program of the EMBOSS package (EMBOSS: The European Molecular Biology Open Software Suite, Rice et al., 2000 Trends in Genetics 16:276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm as implemented in the Needle program of the EMBOSS package, preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

### Homologous Sequence:

The term “homologous sequence” is defined herein as a predicted protein having an E value (or expectancy score) of less than 0.001 in a tfasty search (Pearson, W. R. 1999 in Bioinformatics Methods and Protocols, S. Misener and S. A. Krawetz, ed., pp. 185-219) with the CALB polypeptide having lipolytic activity of SEQ ID No: 2, i.e., the mature polypeptide thereof.

### Polypeptide Fragment:

The term “polypeptide fragment” is defined herein as a polypeptide having one or more (several) amino acids deleted from the amino and/or carboxyl terminus of the mature polypeptide of SEQ ID No: 2; or a homologous sequence thereof; wherein the fragment has lipolytic activity.

### Subsequence:

The term “subsequence” is defined herein as a nucleotide sequence having one or more (several) nucleotides deleted from the 5′ and/or 3′ end of the mature polypeptide coding sequence of SEQ ID No: 1; or a homologous sequence thereof; wherein the subsequence encodes a polypeptide fragment having lipollytic activity.

### Allelic Variant:

The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

### Isolated Polynucleotide:

The term “isolated polynucleotide” as used herein refers to a polynucleotide that is isolated from a source. In a preferred aspect, the polynucleotide is at least 1% pure, at least 5% pure, at least 10% pure, at least 20% pure, at least 40% pure, at least 60% pure, at least 80% pure, or at least 90% pure, as determined by agarose electrophoresis.

### Substantially Pure Polynucleotide:

The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, at most 8%, at most 6%, at most 5%, at most 4%, at most 3%, at most 2%, at most 1%, or at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, at least 92% pure, at least 94% pure, at least 95% pure, at least 96% pure, at least 97% pure, at least 98% pure, at least 99% pure, or at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

### Coding Sequence:

the term “coding sequence” is defined herein as a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic nucleotide sequence, or recombinant nucleotide sequence.

cDNA:

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

### Nucleic Acid Construct:

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

### Control Sequences:

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

### Operably Linked:

The term “operably linked” denotes herein a configuration in which a control sequence is placed at an appropriate position relative to the coding sequence of a polynucleotide sequence such that the control sequence directs the expression of the coding sequence of a polypeptide.

### Expression:

The term “expression” includes any step involved in the production of a polypeptide including, but not limited to, transcription, post-transcriptional modification, translation, post-translational modification, and secretion.

### Expression Vector:

The term “expression vector” is defined herein as a linear or circular DNA molecule that comprises a polynucleotide encoding a polypeptide of the present invention and is operably linked to additional nucleotides that provide for its expression.

### Host Cell:

The term “host cell”, as used herein, includes any cell type that is susceptible to transformation, transfection, transduction, and the like with a nucleic acid construct or expression vector comprising a polynucleotide of the present invention.

### Improved Specific Activity:

The term improved specific activity, as used herein implies increased lipase activity. The lipase activity may be determined using the assay for specific activity (LU assay) described in the “Materials and methods”.

### Improved Transesterification Activity:

The term improved improved transesterification activity, as used herein implies increased activity in the assay for transesterification activity described in the Examples. The assay simulates an industrial process for production of biodiesel.

### Conventions for Designation of Variants

For purposes of the present invention, the amino acid sequence of a polypeptide, i.e., the mature polypeptide disclosed in SEQ ID No: 2, is used to determine the corresponding amino acid residue in another polypeptide. The amino acid sequence of another polypeptide is aligned with the mature polypeptide disclosed in SEQ ID No: 2, and based on the alignment the amino acid position number corresponding to any amino acid residue in the amino acid sequence of the polypeptide disclosed in SEQ ID No: 2 can be determined.

An alignment of polypeptide sequences may be made, for example, using “ClustalW” (Thompson, J D, et al 1994, CLUSTAL W: Improving the sensitivity of progressive multiple sequence alignment through sequence weighting, positions-specific gap penalties and weight matrix choice, Nucleic Acids Research 22:4673-4680). An alignment of DNA sequences may be done using the polypeptide alignment as a template, replacing the amino acids with the corresponding codon from the DNA sequence.

Pairwise sequence comparison algorithms in common use are adequate to detect similarities between polypeptide sequences that have not diverged beyond the point of approximately 20-30% sequence identity (Doolittle, 1992, Protein Sci. 1:191-200; Brenner et al., 1998, PNAS USA 95:6073-6078). However, truly homologous polypeptides with the same fold and similar biological function have often diverged to the point where traditional sequence-based comparison fails to detect their relationship (Lindahl and Elofsson, 2000, J. Mol. Biol. 295:613-615). Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul at al., 1997, Nucleic Acids Res. 25:3389-3402). Even greater sensitivity can be achieved if the family or superfamily for the polypeptide of interest has one or more (several) representatives in the protein structure databases. Programs such as GenTHREADER (Jones 1999, J. Mol. Biol. 287:797-815; McGuffin and Jones, 2003, Bioinformatics 19:874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313:903-919, can be used to align a sequence of unknown structure with the superfamily models present in the SCOP database. These alignments can in turn be used to generate homology models for the polypeptide of interest, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Eng. 11:739-747), and implementations of these algorithms can additionally be utilized to query structure databases with a structure of interest in order to discover possible structural homologs (e.g. Holm & Park, 2000, Bioinformatics 16:566-567). These structural alignments can be used to predict the structurally and functionally corresponding amino acid residues in proteins within the same structural superfamily. This information, along with information derived from homology modeling and profile searches, can be used to predict which residues to mutate when moving mutations of interest from one protein to a close or remote homolog.

In describing the various variants having lipolytic activity of the present invention, the nomenclature described below is adapted for ease of reference. In all cases, the accepted IUPAC single letter or triple letter amino acid abbreviation is employed.

### Substitutions:

For an amino acid substitution, the following nomenclature is used: Original amino acid, position, substituted amino acid. Accordingly, the substitution of threonine with alanine at position 226 is designated as “Thr226Ala” or “T226A”. Multiple mutations are separated by addition marks (“+”), e.g., “Gly205Arg+Ser411Phe” or “G205R+S411-F”, representing mutations at positions 205 and 411 substituting glycine (G) with arginine (R), and serine (S) with phenylalanine (F), respectively.

### Deletions:

For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g., “Gly195*+Ser411” or “G195*+S411*”.

### Insertions:

For an amino acid insertion, the following nomenclature is used: Original amino acid, position, original amino acid, new inserted amino acid. Accordingly the insertion of lysine after glycine at position 195 is designated “Gly195GlyLys” or “G195GK”. Multiple insertions of amino acids are designated [Original amino acid, position, original amino acid, new inserted amino acid #1, new inserted amino acid #2; etc.]. For example, the insertion of lysine and alanine after glycine at position 195 is indicated as “Gly 195GlyLysAla” or “G195GKA”.

In such cases the inserted amino acid residue(s) are numbered by the addition of lower case letters to the position number of the amino acid residue preceding the inserted amino acid residue(s). In the above example the sequences would thus be:

| Parent: | Variant: |
|---|---|
| 195 | 195 195a 195b |
| G | G - K - A |

### Parent Polypeptide

The parent polypeptide may be a fungal polypeptide. In one aspect, the fungal polypeptide is a filamentous fungal polypeptide such as a Candida Antarctica lipase B, Hyphozyma sp. lipase, Ustilago maydis lipase, Giberella zeae lipase (Fusarium graminearum lipase), Debaryomyces hansenii lipase, Aspergillus fumigates lipase, Aspergillus oryzae lipase, or Neurospora crassa lipase polypeptide. It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

In one aspect, the parent polypeptide consists or comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to is a lipase selected from the group consisting of: Candida Antarctica lipase B (SEQ ID No: 2), Hyphozyma sp. lipase (SEQ ID No: 3), Ustilago maydis lipase (SEQ ID No: 4), Giberella zeae lipase (Fusarium graminearum lipase, SEQ ID No: 5), Debaryomyces hansenii lipase (SEQ ID No: 6), Aspergillus fumigates lipase (SEQ ID No: 7), Aspergillus oryzae lipase (SEQ ID No: 8), and Neurospora crassa lipase (SEQ ID No: 9).

TABLE 1
Lipase amino acid sequences

| SEQ ID | | | Amino |
|---|---|---|---|
| No: | Lipase | Source | acids |
| 2 | Candida antarctica lipase B | UniProt 1TCA | 317 |
| | (CALB) | | |
| 3 | Hyphozyma sp. lipase | WO9324619 | 319 |
| 4 | Ustilago maydis lipase | UniProt Q4pep1 | 336 |
| 5 | Gibberella zeae | UniProt Q4HUY1 | 445 |
| | (Fusarium graminearum) lipase | | |
| 6 | Debaryomyces hansenii lipase | UniProt Q6BVP4 | 455 |
| 7 | Aspergillus fumigates lipase | UniProt Q4WG73 | 440 |
| 8 | Aspergillus oryzae lipase | UniProt Q2UE03 | 401 |
| 9 | Neurospora crassa lipase | UniProt Q7RYD2 | 388 |

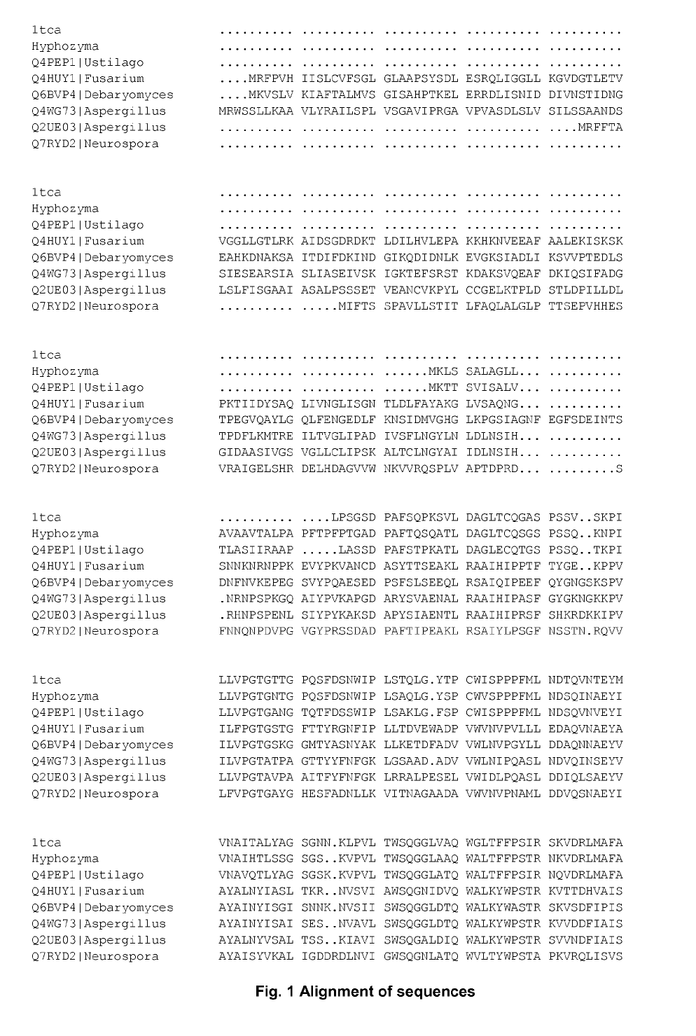

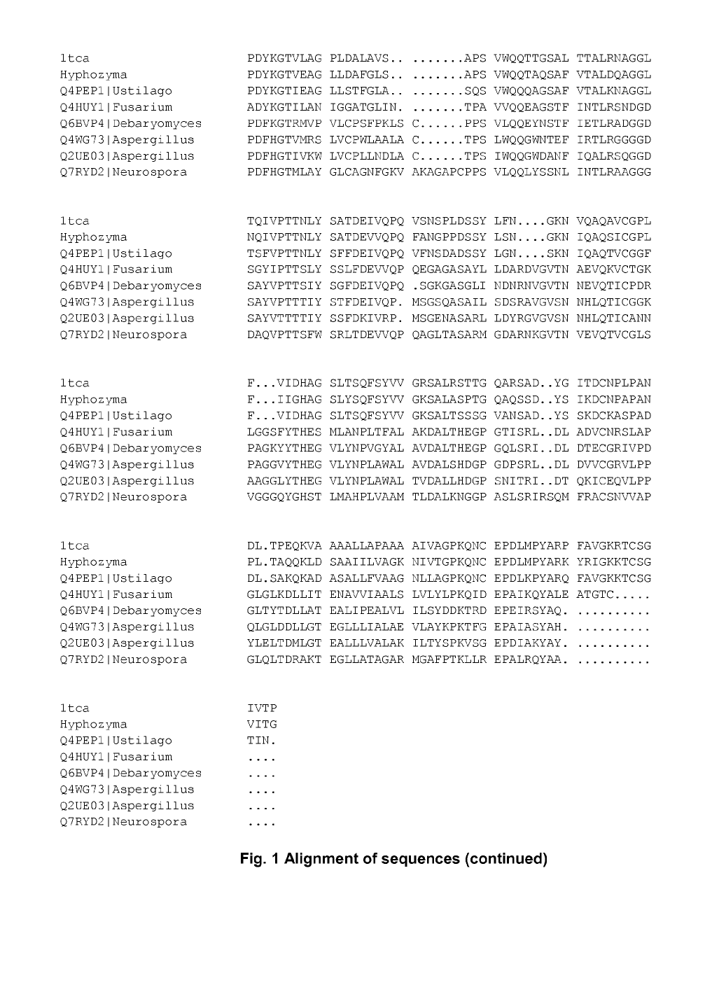

Alignment of the lipases from table 1 is shown in FIG. 1 and was done using the needle program from the EMBOSS package (http://www.emboss.org) version 2.8.0 with the following parameters: Gap opening penalty: 10.00, Gap extension penalty: 0.50, Substitution matrix: EBLOSUM62. The software is described in EMBOSS: The European Molecular Biology Open Software Suite (2000), Rice, P. Longden, I. and Bleasby, A., Trends in Genetics 16(6):276-277. The program needle implements the global alignment algorithm described in Needleman, S. B. and Wunsch, C. D. (1970) J. Mol. Biol. 48:443-453, and Kruskal, J. B. (1983). Other parent polypeptides may aligned to the sequences in FIG. 1 by the same method or by the methods described in D. Sankoff and J. B. Kruskal, (ed.), Time warps, string edits and macromolecules: the theory and practice of sequence comparison, pp. 1-44 Addison Wesley.

In one aspect, the parent polypeptide has an amino acid sequence that differs with less than 20 amino acids, less than 19 amino acids, less than 18 amino acids, less than 17 amino acids, less than 16 amino acids, less than 15 amino acids, less than 14 amino acids, less than 13 amino acids, less than 12 amino acids, less than 11 amino acids, less than 10 amino acids, less than 9 amino acids, less than 8 amino acids, less than 7 amino acids, less than 6 amino acids, less than 5 amino acids, less than 4 amino acids, less than 3 amino acids, less than 2 amino acids, or with 0 amino acid from the polypeptides of any of SEQ ID No: 2, 3, 4, 5, 6, 7, 8 and/or 9.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, S— and 4-methylproline, and 3,3-dimethylproline.

In one aspect the parent polypeptide consists or comprises the amino acid sequences of any of SEQ ID No: 2, 3, 4, 5, 6, 7, 8 and/or 9, allelic variants thereof or fragments thereof. In one aspect, the parent polypeptide consists or comprises the mature polypeptides of any of SEQ ID No: 2, 3, 4, 5, 6, 7, 8 and/or 9. In another aspect, the parent polypeptide consists or comprises amino acids selected from the group consisting of: amino acid 1 to 317 of SEQ ID No: 2; amino acid 1 to 319 of SEQ ID No: 3; amino acid 1 to 336 of SEQ ID No: 4; amino acid 1 to 445 of SEQ ID No: 5; amino acid 1 to 455 of SEQ ID No: 6; amino acid 1 to 440 of SEQ ID No: 7; amino acid 1 to 401 of SEQ ID No: 8; amino acid 1 to 388 of SEQ ID No: 9; allelic variants thereof and fragments thereof.

An allelic variant of the polypeptide or the mature polypeptide of any of SEQ ID No: 2, 3, 4, 5, 6, 7, 8 and/or 9 is a polypeptide encoded by an allelic variant, i.e. any of two or more alternative forms of a gene occupying the same chromosomal locus.

A fragment of the polypeptide or the mature polypeptide of any of SEQ ID No: 2, 3, 4, 5, 6, 7, 8 and/or 9 is a polypeptide having one or more (several) amino acids deleted from the amino and/or carboxyl terminus of this amino acid sequence. Preferably, a fragment contains at least 200 amino acid residues, at least 210 amino acid residues, or at least 220 amino acid residues.

A subsequence of the mature polypeptide coding sequence of SEQ ID No: 1, or a homolog thereof, is a nucleotide sequence where one or more (several) nucleotides have been deleted from the 5′- and/or 3′-end. Preferably, a subsequence contains at least 600 nucleotides, at least 630 nucleotides, or at least 660 nucleotides.

The polynucleotide of SEQ ID No: 1; or a subsequence thereof; as well as the amino acid sequence of SEQ ID No: 2; or a fragment thereof; may be used to design nucleic acid probes to identify and clone DNA encoding parent polypeptides from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, at least 25, at least 35, or at least 70 nucleotides in length. It is however, preferred that the nucleic acid probe is at least 100 nucleotides in length. For example, the nucleic acid probe may be at least 200 nucleotides, at least 300 nucleotides, at least 400 nucleotides, at least 500 nucleotides, or at least 600 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may be screened for DNA that hybridizes with the probes described above and encodes a parent polypeptide. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID No: 1, or a subsequence thereof, the carrier material is used in a Southern blot. For purposes of the present invention, hybridization indicates that the polynucleotide hybridizes to a labeled nucleotide probe corresponding to the polynucleotide shown in SEQ ID No: 1, its complementary strand, or a subsequence thereof, under low to very high stringency conditions. Molecules to which the probe hybridizes can be detected using, for example, X-ray film or any other detection means known in the art.

In one aspect, the nucleic acid probe is the mature polypeptide coding sequence of SEQ ID No: 1. In another aspect, the nucleic acid probe is nucleotides 76 to 1026 of SEQ ID No: 1. In another aspect, the nucleic acid probe is a polynucleotide sequence that encodes the polypeptide of SEQ ID No: 2, or a subsequence thereof.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), or at 70° C. (very high stringency).

For short probes that are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, PNAS USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes that are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a third aspect, the parent polypeptide is encoded by a polynucleotide comprising or consisting of a nucleotide sequence with a degree of identity to the mature polypeptide coding sequence of SEQ ID No: 1 of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In one aspect, the mature polypeptide coding sequence is nucleotides 76 to 1026 of SEQ ID No: 1.

The parent polypeptide may be obtained from microorganisms of any genus. For purposes of the present invention, the term “obtained from” as used herein in connection with a given source shall mean that the parent polypeptide encoded by a polynucleotide is produced by the source or by a cell in which the polynucleotide from the source has been inserted. In one aspect, the parent polypeptide is secreted extracellularly.

### Preparation of Variants

Variants of a parent polypeptide can be prepared according to any mutagenesis procedure known in the art, such as site-directed mutagenesis, synthetic gene construction, semi-synthetic gene construction, random mutagenesis, shuffling, etc.

Site-directed mutagenesis is a technique in which one or several mutations are created at a defined site in a polynucleotide molecule encoding the parent polypeptide. The technique can be performed in vitro or in vivo.

Synthetic gene construction entails in vitro synthesis of a designed polynucleotide molecule to encode a polypeptide molecule of interest. Gene synthesis can be performed utilizing a number of techniques, such as the multiplex microchip-based technology described in Tian, et. al., Nature 432:1050-1054, and similar technologies wherein olgionucleotides are synthesized and assembled upon photo-programable microfluidic chips.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent polypeptide and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, for example, Scherer & Davis 1979 PNAS USA 76:4949-4955; and Barton et al. 1990 Nucleic Acids Research 18:7349-4966.

Site-directed mutagenesis can be accomplished in vivo by methods known in the art. See, for example, U.S. Patent Application Publication 2004/0171154; Storici et al. 2001 Nature Biotechnology 19:773-776; Kren et al. 1998 Nat. Med. 4:285-290; and Calissano & Macino 1996 Fungal Genet. Newslett. 43:15-16.

Any site-directed mutagenesis procedure can be used in the present invention. There are many commercial kits available that can be used to prepare variants of a parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson & Sauer 1988 Science 241:53-57; Bowie & Sauer 1989 PNAS USA 86:2152-2156; WO95/17413; or WO95/22625. Other methods that can be used include error-prone PCR, phage display (e.g., Lowman et al. 1991 Biochem. 30:10832-10837; U.S. Pat. No. 5,223,409; WO92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplfied using site-specific mutagenic primers, while yet other regions may be subjected to error-prone PCR or non-error prone PCR ampflication. Polynucleotide fragments may then be shuffled.

Alternatively, a variant and polynucleotide thereof may be isolated from nature using standard techniques, such as the techniques disclosed herein for isolating a parent polypeptide and polynucleotide thereof.

### Variants

In a second aspect the present invention relates to an isolated variant of a parent polypeptide, wherein said variant is: (a) a polypeptide comprising a substitution at at least one amino acid residue at a position corresponding to any of positions: 41, 83, 129, 207 or 284 of SEQ ID No: 2, wherein said variant has lipolytic activity, which variant compared to the parent polypeptide has improved stability, and has an amino acid sequence with at least 60% identity to the mature polypeptide of SEQ ID No: 2; (b) a polypeptide encoded by a polynucleotide that hybridizes under at least low stringency conditions with (i) the mature polypeptide coding sequence of SEQ ID No: 1, (ii) the genomic DNA sequence comprising the mature polypeptide coding sequence of SEQ ID No: 1, or (iii) a full-length complementary strand of (i) or (ii); or (c) a polypeptide encoded by a polynucleotide comprising a nucleotide sequence having at least 60% identity with the mature polypeptide coding sequence of SEQ ID No: 1.

In one aspect, the variant is encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID No: 1, (ii) the genomic DNA sequence comprising the mature polypeptide coding sequence of SEQ ID No: 1, (iii) a subsequence of (i) or (ii), or (iv) a full-length complementary strand of (i), (ii), or (iii) (J. Sambrook, E. F. Fritsch, and T. Maniatis, 1989, Molecular Cloning, A Laboratory Manual, 2d edition, Cold Spring Harbor, N.Y.). The subsequence may encode a polypeptide fragment having lipolytic activity. In one aspect, the complementary strand is the full-length complementary strand of the mature polypeptide coding sequence of SEQ ID No: 1.

In one aspect the invention relates to a variant, wherein the parent polypeptide consists or comprises an amino acid sequence with at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to is a lipase selected from the group consisting of: Candida Antarctica lipase B (SEQ ID No: 2), Hyphozyma sp. lipase (SEQ ID No: 3), Ustilago maydis lipase (SEQ ID No: 4), Giberella zeae lipase (Fusarium graminearum lipase, SEQ ID No: 5), Debaryomyces hansenii lipase (SEQ ID No: 6), Aspergillus fumigates lipase (SEQ ID No: 7), Aspergillus oryzae lipase (SEQ ID No: 8), and Neurospora crassa lipase (SEQ ID No: 9).

In one aspect, the variant comprises a substitution of at least one amino acid residue at a position corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys as one or more (several) substitutions at positions corresponding to positions 41, 83, 129, 207 or 284 of the polypeptide of SEQ ID No: 2, respectively.

In one aspect, the variant comprises a substitution at a position corresponding to position 41 of the polypeptide of SEQ ID No: 2. In another aspect, the variant comprising a substitution at a position corresponding to position 41 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Ala as a substitution at a position corresponding to position 41 of the polypeptide of SEQ ID No: 2. In another aspect, the variant comprises the substitution G41A of the polypeptide of SEQ ID No: 2.

In one aspect, the variant comprises a substitution at a position corresponding to position 83 of the polypeptide of SEQ ID No: 2. In another aspect, the variant comprises a substitution at a position corresponding to position 83 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Leu as a substitution at a position corresponding to position 83 of the polypeptide of SEQ ID No: 2. In another aspect, the variant comprises the substitution M83L of the polypeptide of SEQ ID No: 2.

In one aspect, the variant comprises a substitution at a position corresponding to position 129 of the polypeptide of SEQ ID No: 2. In one aspect, the variant comprises a substitution at a position corresponding to position 129 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Leu as a substitution at a position corresponding to position 129 of the polypeptide of SEQ ID No: 2. In another aspect, the variant comprises the substitution M129L of the polypeptide of SEQ ID No: 2.

In one aspect, the variant comprises a substitution at a position corresponding to position 207 of the polypeptide of SEQ ID No: 2. In another aspect, the variant comprises a substitution at a position corresponding to position 207 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Ala as a substitution at a position corresponding to position 207 of the polypeptide of SEQ ID No: 2. In another aspect, the variant comprises the substitution G207A of the polypeptide of SEQ ID No: 2.

In one aspect, the variant comprises a substitution at a position corresponding to position 284 of the polypeptide of SEQ ID No: 2. In another aspect, the variant comprises a substitution at a position corresponding to position 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises Ala as a substitution at a position corresponding to position 284 of the polypeptide of SEQ ID No: 2. In another aspect, the variant comprises the substitution A284N of the polypeptide of SEQ ID No: 2.

In another aspect, the variant comprises a combination of 2 substitutions, 3 substitutions, 4 substitutions, or 5 substitutions at positions corresponding to any of positions 41, 83, 129, 207 or 284 of the polypeptide of SEQ ID No: 2.

In another aspect, the variant comprises a combination of two substitutions at positions corresponding to any of positions 41, 83, 129, 207 or 284 of the polypeptide of SEQ ID No: 2. In another aspect, the variant comprises a combination of two substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises a combination of two substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the variant comprises a combination of two substitutions of any of G41A, M83L, M129L, or G207A of the polypeptide of SEQ ID No: 2.

In another aspect, the variant comprises a combination of three substitutions at positions corresponding to any of positions 41, 83, 129, 207 or 284 of the polypeptide of SEQ ID No: 2. In another aspect, the variant comprises a combination of three substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises a combination of three substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the variant comprises a combination of three substitutions of any of G41A, M83L, M129L, or G207A of the polypeptide of SEQ ID No: 2.

In another aspect, the variant comprises a combination of four substitutions at positions corresponding to any of positions 41, 83, 129, 207 or 284 of the polypeptide of SEQ ID No: 2. In another aspect, the variant comprises a combination of four substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the variant comprises a combination of four substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the variant comprises a combination of four substitutions of any of G41A, M83L, M129L, or G207A of the polypeptide of SEQ ID No: 2.

In one aspect the invention relates to a variant, wherein at least one further position in the amino acid sequence of the parent polypeptide corresponding to any of positions 103, 197, 223 or 278 103, 197, 223 or 278 in SEQ ID No: 2 is substituted.

In one aspect the invention relates to a variant, wherein at least one further position in the amino acid sequence of the parent polypeptide corresponding to any of positions 103, 197, 223 or 278 in SEQ ID No: 2 is substituted with Ala, Arg, Asn, Asp, Cys, Gln, Glu, Gly, His, Ile, Leu, Lys, Met, Phe, Pro, Ser, Thr, Trp, Tyr, or Val. In one aspect, the variant comprises Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys as one or more (several) substitutions at positions corresponding to positions 103, 197, 223 or 278 of the polypeptide of SEQ ID No: 2, respectively.

In one aspect the invention relates to a variant, wherein the substitutions of positions: 41, 83, 103, 129, 197, 207 or 223 of SEQ ID No: 2 are 41A, 83L, 103G, 129L, 197G, 207A, 223G or 278A.

In one aspect the invention relates to a variant, wherein said variant comprise substitutions selected from any of the following: (a) G41A; (b) M83L; (c) M83L+T103G; (d) M83L+T103G+M129L; (e) M83L+T103G+M129L+G207A+D223G; (f) M83L+T103G+M129L+D223G; (g) T103G; (h) T103G+M129L; (i) T103G+M129L+S197G+G207A+D223G; (j) T103G+M129L+G207A; (k) T103G+M129L+G207A+D223G; (l) T103G+M129L+D223G (m) M129L; (n) S197G; (O) G207A; (p) D223G; (q) M83L+T103G+M129L+A148P; (r) N97Q+T103G+M129L; (s) T103G+W104H+ M129L; (t) T103G+M129L+A148P; (u) T103G+M129L+S197G+G207A+D223G P303K; (v) T103G+M129L+G207A+P303K; (w) T103G+M129L+D223G+P303K; (x) T103G+M129L+P303K; (y) T103G+P303K.

In one aspect the invention relates to a variant comprising a substitution in a position selected from the list consisting of 19, 31, 41, 44, 83, 96, 97, 103, 114, 129, 134, 148, 174, 197, 207, 223, 226, 244, 246, 250, 251, 254, 255, 263, 264, 278, 284, 288, 303, and 315.

In one aspect the invention relates to a variant comprising a substitution selected from the list consisting of G19A, S31R, G41A, G411, G41S, G44A, M83L, N96E, N97Q, N97T, T103G, G114A, M129L, D134P, A148P, T174N, S197G, G207A, D223G, G226A, T244P, G246A, S250R, A251P, G254S, G254T, 1255S, 1255T, A263P, N264P, L278A, A284N, G288P, P303K, and V3151.

In one aspect the invention relates to a variant comprising a combination of substitutions selected from the list consisting of (a) P303K+S197G+A284N+D223G+T244P+V3151; (b) M129L+S197G+G207A+D223G+G41A+A148P; (c) T103G+M129L+S197G+G207A+D223G+G41A+; (d) T103G+M129L+S197G+G207A+D223G+G41A+A148P; (e) M129L+D223G+P303K; (f) M129L+G207A+D223G+P303K; (g) M83L+T103G+M129L; (h) M83L+T103G+M129L+A148P; (i) M83L+T103G+M129L+D223G; (j) S197G+A284N+P303K; (k) S197G+T244P+A284N+V3151; (l) T103G+A148P+G207A; (m) T103G+A148P+G207A+S197G; (n) T103G+M129L; (O) T103G+M129L+A148P; (p) T103G+M129L+A251P; (q) T103G+M129L+D223G; (r) T103G+M129L+D223G+G207A; (s) T103G+M129L+D223G+G207A+M83L; (t) T103G+M129L+D223G+G207A+M83L+A263P; (u) T103G+M129L+D223G+G207A+M83L+D134P; (v) T103G+M129L+D223G+G207A+M83L+G114A; (w) T103G+M129L+D223G+G207A+M83L+G19A; (x) T103G+M129L+D223G+G207A+M83L+G226A; (y) T103G+M129L+D223G+G207A+M83L+G246A; (z) T103G+M129L+D223G+G207A+M83L+G288P; (aa) T103G+M129L+D223G+G207A+M83L+G41A+L278A; (ab) T103G+M129L+D223G+G207A+M83L+G41S; (ac) T103G+M129L+D223G+G207A+M83L+G44A; (ad) T103G+M129L+D223G+G207A+M83L+N264P; (ae) T103G+M129L+D223G+G207A+M83L+S250R; (af) T103G+M129L+D223G+G207A+M83L+S31R; (ag) T103G+M129L+D223G+G207A+S197G; (ah) T103G+M129L+D223G+G207A+S197G+P303K; (ai) T103G+M129L+D223G+P303K; (aj) T103G+M129L+G207A; (ak) T103G+M129L+G207A+P303K; (al) T103G+M129L+G254S; (am) T103G+M129L+G254T; (an) T103G+M129L+1255S; (ao) T103G+M129L+1255T; (ap) T103G+M129L+N96E; (aq) T103G+M129L+N97Q; (ar) T103G+M129L+N97T; (as) T103G+M129L+P303K; (at) T103G+M129L+P303K+G207A+D223G; and (au) T103G+M129L+P303K+T174N.

In one aspect the invention relates to a variant, wherein said variant has improved properties, such as improved stability in the presence of elevated temperatures, oxidizing conditions and/or alcohol, improved specific activity, and/or improved transesterification activity.

In one aspect the invention relates to a variant, wherein said variant has an amino acid sequence with at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to the mature polypeptide of SEQ ID No: 2.

In one aspect the invention relates to a variant, wherein said variant is encoded by a polynucleotide that hybridizes under at least low stringency conditions, at least medium stringency conditions, at least medium-high stringency conditions, or at least high stringency conditions with (i) the mature polypeptide coding sequence of SEQ ID No: 1, (ii) the genomic DNA sequence comprising the mature polypeptide coding sequence of SEQ ID No: 1, or (iii) a full-length complementary strand of (i) or (ii).

In one aspect the invention relates to a variant, wherein said variant is encoded by a polynucleotide comprising a nucleotide sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity with the mature polypeptide coding sequence of SEQ ID No: 1.

In one aspect the invention relates to a variant, which has improved lipolytic activity.

### Polynucleotides

The present invention also relates to isolated polynucleotides that encode variants of a parent polypeptide, wherein the polynucleotides encode variants comprising a substitution of at least one amino acid residue at a position corresponding to any of positions 41, 83, 129, 207 or 284 of SEQ ID No: 2.

In one aspect, the isolated polynucleotide encodes a variant comprising a substitution of at least one amino acid residue at a position corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In one aspect, the isolated polynucleotide encodes a variant comprising Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys as one or more (several) substitutions at positions corresponding to positions 41, 83, 129, 207 or 284 of the polypeptide of SEQ ID No: 2, respectively.

In one aspect, the isolated polynucleotide encodes a variant comprising a substitution at a position corresponding to position 41 of the polypeptide of SEQ ID No: 2. In one aspect, the isolated polynucleotide encodes a variant comprising a substitution at a position corresponding to position 41 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising Ala as a substitution at a position corresponding to position 41 of the polypeptide of SEQ ID No: 2. In another aspect, the isolated polynucleotide encodes a variant comprising the substitution G41A of the polypeptide of SEQ ID No: 2.

In one aspect, the isolated polynucleotide encodes a variant comprising a substitution at a position corresponding to position 83 of the polypeptide of SEQ ID No: 2. In one aspect, the isolated polynucleotide encodes a variant comprising a substitution at a position corresponding to position 83 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising Leu as a substitution at a position corresponding to position 83 of the polypeptide of SEQ ID No: 2. In another aspect, the isolated polynucleotide encodes a variant comprising the substitution M83L of the polypeptide of SEQ ID No: 2.

In one aspect, the isolated polynucleotide encodes a variant comprising a substitution at a position corresponding to position 129 of the polypeptide of SEQ ID No: 2. In one aspect, the isolated polynucleotide encodes a variant comprising a substitution at a position corresponding to position 129 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising Leu as a substitution at a position corresponding to position 129 of the polypeptide of SEQ ID No: 2. In another aspect, the isolated polynucleotide encodes a variant comprising the substitution M129L of the polypeptide of SEQ ID No: 2.

In one aspect, the isolated polynucleotide encodes a variant comprising a substitution at a position corresponding to position 207 of the polypeptide of SEQ ID No: 2. In one aspect, the isolated polynucleotide encodes a variant comprising a substitution at a position corresponding to position 207 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising Ala as a substitution at a position corresponding to position 207 of the polypeptide of SEQ ID No: 2. In another aspect, the isolated polynucleotide encodes a variant comprising the substitution G207A of the polypeptide of SEQ ID No: 2.

In another aspect, the isolated polynucleotide encodes a variant comprising a combination of 2 substitutions, 3 substitutions, or 4 substitutions at positions corresponding to any of positions 41, 83, 129, 207 or 284 of the polypeptide of SEQ ID No: 2.

In another aspect, the isolated polynucleotide encodes a variant comprising a combination of two substitutions at positions corresponding to any of positions 41, 83, 129, 207 or 284 of the polypeptide of SEQ ID No: 2. In another aspect, the isolated polynucleotide encodes a variant comprising a combination of two substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising a combination of two substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the isolated polynucleotide encodes a variant comprising a combination of two substitutions of any of G41A, M83L, M129L, or G207A of the polypeptide of SEQ ID No: 2.

In another aspect, the isolated polynucleotide encodes a variant comprising a combination of three substitutions at positions corresponding to any of positions 41, 83, 129, 207 or 284 of the polypeptide of SEQ ID No: 2. In another aspect, the isolated polynucleotide encodes a variant comprising a combination of three substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising a combination of three substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the isolated polynucleotide encodes a variant comprising a combination of three substitutions of any of G41A, M83L, M129L, or G207A of the polypeptide of SEQ ID No: 2.

In another aspect, the isolated polynucleotide encodes a variant comprising a combination of four substitutions at positions corresponding to any of positions 41, 83, 129, 207 or 284 of the polypeptide of SEQ ID No: 2. In another aspect, the isolated polynucleotide encodes a variant comprising a combination of four substitutions at positions corresponding to any of positions 41, 83, 129, 207 or 284 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In another aspect, the isolated polynucleotide encodes a variant comprising a combination of four substitutions of any of Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys at positions corresponding to positions 41, 83, 129, 207 or 284, respectively, of the polypeptide of SEQ ID No: 2. In another aspect, the isolated polynucleotide encodes a variant comprising a combination of four substitutions of any of G41A, M83L, M129L, or G207A of the polypeptide of SEQ ID No: 2.

In addition to the substitutions at any of the positions 41, 83, 129, 207 or 284 of the polypeptide of SEQ ID No: 2, the isolated polynucleotide encodes a variant that may comprise further substitutions. In one aspect, the isolated polynucleotide encodes a variant further comprising a substitution of at least one amino acid residue at a position corresponding to any of positions 103, 197, 223 or 278 of the polypeptide of SEQ ID No: 2 with Ala, Arg, Asn, Asp, Cys, Gln, Glu, Gly, His, Ile, Leu, Lys, Met, Phe, Pro, Ser, Thr, Trp, Tyr, or Val. In one aspect, the isolated polynucleotide encodes a variant comprising Trp, Tyr or Phe or Trp, Trp, Tyr or Phe or Trp, Thr, Tyr or Phe or Trp, Asp, Ser, His, Ile, Asn, Asp, Asp, Asn, Pro, Asp, Ser, Tyr or Phe or Trp, and Lys as one or more (several) substitutions at positions corresponding to positions 103, 197, 223 or 278 of the polypeptide of SEQ ID No: 2, respectively.

In one aspect the isolated polynucleotide encodes a variant, wherein the substitutions of positions: 41, 83, 103, 129, 197, 207 or 223 of SEQ ID No: 2 are 41A, 83L, 103G, 129L, 197G, 207A, 223G or 278A.

In an embodiment of any of the preceeding aspects the isolated polynucleotide encodes a variant comprising a substitution in a position selected from the list consisting of 19, 31, 41, 44, 83, 96, 97, 103, 114, 129, 134, 148, 174, 197, 207, 223, 226, 244, 246, 250, 251, 254, 255, 263, 264, 278, 284, 288, 303, and 315.

In an embodiment of any of the preceeding aspects the isolated polynucleotide encodes a variant comprising a substitution selected from the list consisting of G19A, S31R, G41A, G411, G41S, G44A, M83L, N96E, N97Q, N97T, T103G, G114A, M129L, D134P, A148P, T174N, S197G, G207A, D223G, G226A, T244P, G246A, S250R, A251P, G254S, G254T, 1255S, 1255T, A263P, N264P, L278A, A284N, G288P, P303K, and V3151.

In an embodiment of any of the preceeding aspects the isolated polynucleotide encodes a variant comprising a combination of substitutions selected from the list consisting of (a) P303K+S197G+A284N+D223G+T244P+V3151; (b) M129L+S197G+G207A+D223G+G41A+A148P; (c) T103G+M129L+S197G+G207A+D223G+G41A+; (d) T103G+M129L+S197G+G207A+D223G+G41A+A148P; (e) M129L+D223G+P303K; (f) M129L+G207A+D223G+P303K; (g) M83L+T103G+M129L; (h) M83L+T103G+M129L+A148P; (i) M83L+T103G+M129L+D223G; (j) S197G+A284N+P303K; (k) S197G+T244P+A284N+V3151; (l) T103G+A148P+G207A; (m) T103G+A148P+G207A+S197G; (n) T103G+M129L; (O) T103G+M129L+A148P; (p) T103G+M129L+A251P; (q) T103G+M129L+D223G; (r) T103G+M129L+D223G+G207A; (s) T103G+M129L+D223G+G207A+M83L; (t) T103G+M129L+D223G+G207A+M83L+A263P; (u) T103G+M129L+D223G+G207A+M83L+D134P; (v) T103G+M129L+D223G+G207A+M83L+G114A; (w) T103G+M129L+D223G+G207A+M83L+G19A; (x) T103G+M129L+D223G+G207A+M83L+G226A; (y) T103G+M129L+D223G+G207A+M83L+G246A; (z) T103G+M129L+D223G+G207A+M83L+G288P; (aa) T103G+M129L+D223G+G207A+M83L+G41A+L278A; (ab) T103G+M129L+D223G+G207A+M83L+G41S; (ac) T103G+M129L+D223G+G207A+M83L+G44A; (ad) T103G+M129L+D223G+G207A+M83L+N264P; (ae) T103G+M129L+D223G+G207A+M83L+S250R; (af) T103G+M129L+D223G+G207A+M83L+S31R; (ag) T103G+M129L+D223G+G207A+S197G; (ah) T103G+M129L+D223G+G207A+S197G+P303K; (ai) T103G+M129L+D223G+P303K; (aj) T103G+M129L+G207A; (ak) T103G+M129L+G207A+P303K; (al) T103G+M129L+G254S; (am) T103G+M129L+G254T; (an) T103G+M129L+1255S; (ao) T103G+M129L+1255T; (ap) T103G+M129L+N96E; (aq) T103G+M129L+N97Q; (ar) T103G+M129L+N97T; (as) T103G+M129L+P303K; (at) T103G+M129L+P303K+G207A+D223G; and (au) T103G+M129L+P303K+T174N.

### Nucleic Acid Constructs

The present invention also relates to nucleic acid constructs comprising a polynucleotide encoding a variant of the present invention operably linked to one or more (several) control sequences that direct the expression of the coding sequence in a suitable host cell under conditions compatible with the control sequences.

An isolated polynucleotide encoding a variant of the present invention may be manipulated in a variety of ways to provide for expression of the variant. Manipulation of the polynucleotide prior to its insertion into a vector may be desirable or necessary depending on the expression vector. The techniques for modifying polynucleotides utilizing recombinant DNA methods are well known in the art.

The control sequence may be an appropriate promoter sequence, which is recognized by a host cell for expression of the polynucleotide. The promoter sequence contains transcriptional control sequences that mediate the expression of the variant polypeptide having lipolytic activity. The promoter may be any nucleic acid sequence that shows transcriptional activity in the host cell of choice including mutant, truncated, and hybrid promoters, and may be obtained from genes encoding extracellular or intracellular polypeptides either homologous or heterologous to the host cell. Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, PNAS USA 75: 3727-3731), as well as the tac promoter (DeBoer et al., 1983, PNAS USA 80:21-25). Further promoters are described in “Useful proteins from recombinant bacteria” in Scientific American, 1980, 242:74-94; and in Sambrook et al., 1989, supra. Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO00/56900), Fusarium venenatum Daria (WO00/56900), Fusarium venenatum Quinn (WO00/56900), Fusarium oxysporum trypsin-like protease (WO96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof. In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al. 1992 Yeast 8:423-488.

The control sequence may also be a suitable transcription terminator sequence, which is recognized by a host cell to terminate transcription. The terminator sequence is operably linked to the 3′-terminus of the polynucleotide encoding the variant polypeptide having lipolytic activity. Any terminator that is functional in the host cell of choice may be used in the present invention. Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Preferred terminators for yeast host cells are obtained from the genes for Saccharomyces cerevisiae enolase, Saccharomyces cerevisiae cytochrome C(CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′-terminus of the polynucleotide encoding the variant polypeptide having lipolytic activity. Any leader sequence that is functional in the host cell of choice may be used in the present invention. Preferred leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polypeptide-encoding sequence and, when transcribed, is recognized by the host cell as a signal to add polyadenosine residues to transcribed mRNA. Any polyadenylation sequence that is functional in the host cell of choice may be used in the present invention. Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo & Sherman 1995 Molecular Cellular Biology 15:5983-5990.

The control sequence may also be a signal peptide coding region that codes for an amino acid sequence linked to the amino terminus of a variant polypeptide having lipolytic activity and directs the encoded polypeptide into the cell's secretory pathway. The 5′-end of the coding sequence of the polynucleotide may inherently contain a signal peptide coding region naturally linked in translation reading frame with the segment of the coding region that encodes the secreted variant polypeptide having lipolytic activity. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant polypeptide having lipolytic activity. However, any signal peptide coding region that directs the expressed polypeptide into the secretory pathway of a host cell of choice may be used in the present invention. Effective signal peptide coding sequences for bacterial host cells are the signal peptide coding sequences obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen & Palva 1993 Microbiological Reviews 57:109-137.

Effective signal peptide coding sequences for filamentous fungal host cells are the signal peptide coding sequences obtained from the genes for Aspergillus olyzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding region that codes for an amino acid sequence positioned at the amino terminus of a variant polypeptide having lipolytic activity. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Saccharomyces cerevisiae alpha-factor, Rhizomucor miehei aspartic proteinase, and Myceliophthora thermophila laccase (WO95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences that allow the regulation of the expression of the variant polypeptide having lipolytic activity relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and tip operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant polypeptide having lipolytic activity would be operably linked with the regulatory sequence.

### Expression Vectors

The present invention also relates to recombinant expression vectors comprising a polynucleotide encoding a variant polypeptide of the present invention, a promoter, and transcriptional and translational stop signals. The various nucleotide and control sequences described above may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of the polynucleotide. The choice of the vector will typically depend on the compatibility of the vector with the host cell into which the vector is to be introduced. The vectors may be linear or closed circular plasmids. The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, chloramphenicol, or tetracycline resistance. Suitable markers for yeast host cells are ADE2, HIS3, LEU2, LYS2, METS, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bargene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo. Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6. Examples of origins of replication useful in a filamentous fungal cell are AMA1 and ANS1 (Gems et al. 1991 Gene 98:61-67; Cullen et al. 1987 Nucleic Acids Research 15:9163-9175; WO00/24883). Isolation of the AMA1 gene and construction of plasmids or vectors comprising the gene can be accomplished according to the methods disclosed in WO00/24883.

More than one copy of a polynucleotide of the present invention may be inserted into a host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding a variant polypeptide, which are advantageously used in the recombinant production of the variant. A vector comprising a polynucleotide of the present invention is introduced into a host cell so that the vector is maintained as a chromosomal integrant or as a self-replicating extra-chromosomal vector as described earlier. The choice of a host cell will to a large extent depend upon the gene encoding the polypeptide and its source. The host cell may be any cell useful in the recombinant production of a variant polypeptide having lipolytic activity, e.g., a prokaryote or a eukaryote.

The prokaryotic host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, llyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In a preferred aspect, the bacterial host cell is a Bacillus amyloliquefaciens, Bacillus lentus, Bacillus ficheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausfi cell. In another more preferred aspect, the bacterial host cell is a Bacillus licheniformis cell. In another more preferred aspect, the bacterial host cell is a Bacillus subtilis cell.

The bacterial host cell may also be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus cells. In a preferred aspect, the bacterial host cell is a Streptococcus equisimilis cell. In another preferred aspect, the bacterial host cell is a Streptococcus pyogenes cell. In another preferred aspect, the bacterial host cell is a Streptococcus uberis cell. In another preferred aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans cells. In a preferred aspect, the bacterial host cell is a Streptomyces achromogenes cell. In another preferred aspect, the bacterial host cell is a Streptomyces avermitilis cell. In another preferred aspect, the bacterial host cell is a Streptomyces coelicolor cell. In another preferred aspect, the bacterial host cell is a Streptomyces griseus cell. In another preferred aspect, the bacterial host cell is a Streptomyces lividans cell.

The introduction of DNA into a Bacillus cell may, for instance, be effected by protoplast transformation (see, e.g., Chang & Cohen 1979 Molecular General Genetics 168:111-115), by using competent cells (see, e.g., Young & Spizizen 1961 Journal of Bacteriology 81:823-829, or Dubnau & Davidoff-Abelson 1971 Journal of Molecular Biology 56:209-221), by electroporation (see, e.g., Shigekawa & Dower 1988 Biotechniques 6:742-751), or by conjugation (see, e.g., Koehler & Thorne 1987 Journal of Bacteriology 169:5271-5278). The introduction of DNA into an E coli cell may, for instance, be effected by protoplast transformation (see, e.g., Hanahan 1983 J. Mol. Biol. 166:557-580) or electroporation (see, e.g., Dower et al. 1988 Nucleic Acids Res. 16:6127-6145). The introduction of DNA into a Streptomyces cell may, for instance, be effected by protoplast transformation and electroporation (see, e.g., Gong et al. 2004 Folia Microbiol. (Praha) 49:399-405), by conjugation (see, e.g., Mazodier et al. 1989 J. Bacteriol. 171:3583-3585), or by transduction (see, e.g., Burke et al. 2001 PNAS USA 98:6289-6294). The introduction of DNA into a Pseudomonas cell may, for instance, be effected by electroporation (see, e.g., Choi et al. 2006 J. Microbiol. Methods 64:391-397) or by conjugation (e.g., Pinedo & Smets 2005 Appl. Environ. Microbiol. 71:51-57). Introduction of DNA into a Streptococcus cell may, for instance, be effected by natural competence (see, e.g., Perry & Kuramitsu 1981 Infect. Immun. 32:1295-1297), by protoplast transformation (see, e.g., Catt & Jollick 1991 Microbios. 68:189-207, by electroporation (e.g., Buckley et al. 1999 Appl. Environ. Microbiol. 65:3800-3804) or by conjugation (e.g., Clewell 1981 Microbiol. Rev. 45:409-436). However, any method known in the art for introducing DNA into a host cell can be used.

The host cell may also be a eukaryote, such as a mammalian, insect, plant, or fungal cell. In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980). In an even more preferred aspect, the yeast host cell is a Candida, Hansenula, Kluyveromyces, Pichia, Saccharomyces, Schizosaccharomyces, or Yarrowia cell. In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In an even more preferred aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, or Trichoderma cell. In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP238023 and Yelton et al., 1984, PNAS USA 81:1470-1474. Suitable methods for transforming Fusarium species are described by Malardier et al. 1989 Gene 78:147-156, and WO96/00787. Yeast may be transformed using the procedures described by Becker and Guarente, In Abelson, J. N. and Simon, M. I., editors, Guide to Yeast Genetics and Molecular Biology, Methods in Enzymology, 194:182-187, Academic Press, Inc., New York; Ito et al., 1983, Journal of Bacteriology 153:163; and Hinnen et al., 1978, PNAS USA 75:1920.

### Methods of Production

The present invention also relates to methods of producing a variant polypeptide, comprising: (a) cultivating a host cell, as described herein, under conditions conducive for production of the variant polypeptide; and (b) recovering the variant polypeptide from the cultivation medium.

In the production methods of the present invention, the host cells are cultivated in a nutrient medium suitable for production of the variant polypeptide having lipolytic activity using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. In an alternative aspect, the variant polypeptide is not recovered, but rather a host cell of the present invention expressing a variant is used as a source of the variant.

The variant polypeptide may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein in the Examples.

The resulting variant polypeptide may be recovered by methods known in the art. See, for example, WO05/074647, WO05/074656, and WO07/089,290.

A variant of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson & Lars Ryden, editors, VCH Publishers, New York, 1989) to obtain substantially pure variants having lipolytic activity.

### Compositions

The present invention also relates to compositions comprising a variant having lipolytic activity of the present invention. The polypeptide compositions may be prepared in accordance with methods known in the art. Examples are given below of preferred uses of the variant compositions of the invention. The dosage of the composition of the invention and other conditions under which the composition is used may be determined on the basis of methods known in the art.

### Uses

The present invention is also directed to methods for using the variants having lipolytic activity.

In one aspect the invention relates to a method of stabilizing a lipase catalyzed reaction comprising use of the variant having lipolytic activity of the present invention.

In one aspect the invention relates to a method of perfoming a lipase catalyzed reaction comprising bringing the reactants into contact with the variant having lipolytic activity of the present invention, wherein the reaction is: (a) hydrolysis with a carboxylic acid ester and water as reactants, and a free carboxylic acid and an alcohol as products; (b) ester synthesis with a free carboxylic acid and an alcohol as reactants, and a carboxylic acid ester as product; (c) alcoholysis with a carboxylic acid ester and an alcohol as reactants and as products; or (d) acidolysis with a carboxylic acid ester and a free fatty acid as reactants and as products.

In one aspect the present invention is directed to to methods for using the variants in the production of fatty acid ethyl esters (FAEE) or fatty acid methyl ester (FAME). Such esters are used as e.g. biodiesel, and may be prepared from several types of vegetable oils. Examples of plants which may serve as feed stock for vegetable oils for use as substrate in the production of fatty acid ethyl esters are such as babassu, borage, canola, coconut, corn, cotton, hemp, jatropha, karanj, mustard, oil palm, peanut, rapeseed, rice, soybean, and sunflower.

Microalgae is also considered as feed stock in the production of biodiesel due to the higher photosynthetic efficiency of microalgae in comparison with plants and hence a potentially higher productivity per unit area.

Alternatively, fatty acid methyl esters or fatty acid ethyl esters may be prepared from non-vegetable feed stocks like animal fat such as lard, tallow, butterfat and poultry; or marine oils such as tuna oil and hoki liver oil.

Waste oil can be used as raw material for the production of biodiesel. Fresh vegetable oil and its waste differ in their content of water and free fatty acid. Unlike the conventional chemical routes for synthesis of diesel fuels, biocatalytic routes permit one to carry out the transesterification of a wide variety of oil feed stocks in the presence of acidic impurities, such as free fatty acids. Accordingly, fatty acid distillates (from deodorizer/fatty acid stripping), acid oils (from soap stock splitting in chemical oil refining), waste oils and used oils may serve as feed stock in the production of biodiesel.

Thus, the feed stock can be of crude quality or further processed (refined, bleached and deodorized). Suitable oils and fats may be pure triglyceride or a mixture of triglyceride, diglyceride, monoglyceride, and free fatty acids, commonly seen in waste vegetable oil and animal fats. The feed stock may also be obtained from vegetable oil deodorizer distillates. The type of fatty acids in the feed stock comprises those naturally occurring as glycerides in vegetable and animal fats and oils. These include oleic acid, linoleic acid, linolenic acid, palmetic acid and lauric acid to name a few. Minor constituents in crude vegetable oils are typically phospholipids, free fatty acids and partial glycerides i.e. mono- and diglycerides.

### EXAMPLES

The present invention is further described by the following examples that should not be construed as limiting the scope of the invention.

### Materials and Methods

Chemicals used as buffers and substrates were commercial products of at least reagent grade.

### Determination of Lipolytic Activity (LU)

The lipolytic activity may be determined in Lipase Units (LU). The LU activity is determined using tributyrine as substrate. This method is based on the hydrolysis of tributyrin by the enzyme, and the alkali consumption is registered as a function of time in a pH-stat titration.

One Lipase Unit (LU) is defined as the amount of enzyme which, under standard conditions (i.e. at 30.0° C.; pH 7.0; with 0.12% Gum Arabic as emulsifier and 0.16 M tributyrine as substrate) liberates 1 micromol titrable butyric acid per minute.

A folder AF 95/5 describing this analytical method in more detail is available upon request to Novo Nordisk NS, Denmark, which folder is hereby included by reference.

### Example 1

### Procedure for Variant Generation

The gene of CalB (SEQ ID No: 1) was cloned into an expression vector. A PCR-based site-directed mutagenesis (SDM) was carried out to generate variants of the gene by introducing mutations at specific sites. SDM was carried out using a single mutagenic primer of 20-30 base pairs with the desired amino acid change (substitution/deletion/insertion). Primers used for the mutagenesis were designed such that the mutation lies in the middle of the oligonucleotide with sufficient flanking residues (9-12 basepairs). During the PCR reaction, the primers generated mutant single-stranded DNA. The PCR product was then treated with DpnI restriction enzyme for 6 hours in a PCR machine at 37° C. DpnI digested the methylated or the parental template DNA whereas the newly formed mutated DNA strands that were non-methylated remain intact. The intact newly synthesized double-stranded mutant PCR product was then used to transform competent Escherichia coli cells. Plasmid DNA was isolated from a single isolated transformant and sent for sequence analysis, which confirmed the presence of the desired mutation.

The polymerase used for the PCR reaction was Phusion DNA polymerase (Finnzymes, Cat. No.: F530L). DpnI is from New England Biolabs (Cat. No.: R0176S). The PCR machine is from Applied Biosystems (Model no. GeneAmp9700). Plasmid DNA is isolated using Sigma GenElute Plasmid Miniprep Kit (Cat. No.: PLN350-1KT).

### Example 2

### Thermostability

CalB variants with improved thermostability were identified by measuring the residual activity after incubation at various temperatures.

The thermostability assay was performed by preparing a 0.5 mg/ml purified lipase solution in a 50 mM Tris-Acetate buffer pH 7.0 (Trizma base Sigma T6066) and acetic acid (Merck-60006325001730). Aliquots of 50 microliter of this enzyme solution were placed in PCR tubes (Axygen-PCR-08S-CPC) and incubated in a PCR machine (Applied Biosystem-Geneamp PCR System 9700) at different temperatures (4° C. and 60° C.) for 20 minutes. The temperature treated samples were immediately stored at 4° C. until use.

For preparing the reaction mixture, the temperature treated enzyme samples were diluted sixteen times using 50 mM hepes (Sigma H3375) buffer containing 10 mM Calcium chloride (Sigma C5080) and 0.4% Triton X-100 (Sigma T8787) pH 7.0. Aliquots of 20 microliter of diluted enzyme sample was transferred to a 96 well plate (Nunc-96-F Micro well plate 269620) and to this 80 microliter of 50 mM hepes (Sigma H3375) buffer containing 10 mM Calcium chloride (Sigma C5080) and 0.4% Triton X-100 (Sigma T8787) pH 7.0 were added. A pNP Butyrate substrate solution was prepared by solubilizing 2 mM 4-nitrophenyl butyrate (Sigma N9876) in a 50 mM hepes (Sigma H3375) buffer containing 10 mM Calcium chloride (Sigma C5080) and 0.4% Triton X-100 (Sigma T8787) pH 7.0. Aliquots of 100 microliter substrate solution were added to the 100 microliter diluted enzyme solution.

The reaction mixtures were incubated at 25° C. and absorbances were measured at 405 nm for 5 minutes using a Microtiter Plate Reader (Spectra Max M5, Molecular Devices).

The residual activity percentage (RA %) is calculated as (the activity after 20 min at 60° C. divided by the activity after 20 min at 4° C. and multiplied by 100) %.

TABLE 2
Thermostability of CALB variants. Residual
activity after 20 min incubation at 60° C.

| Variant | RA % |
|---|---|
| CalB wild type | 13 |
| M83L | 0 |
| T103G | 19 |
| M129L | 0 |
| S197G | 0 |
| D134P | 48 |
| G19A | 0 |
| G207A | 0 |
| D223G | 0 |
| G226A | 10 |
| P303K | 0 |
| T244P | 0 |
| S31R | 19 |
| A284N | 0 |
| G114A | 10 |
| S250R | 12 |
| G246A | 9 |
| A263P | 11 |
| V315I | 0 |
| M83L T103G M129L G207A D223G G226A | 103 |
| T103G M129L S197G G207A D223G P303K | 98 |
| M83L T103G M129L D134P G207A D223G | 98 |
| M83L T103G M129L G207A D223G | 96 |
| T103G M129L S197G G207A D223G | 95 |
| G19A M83L T103G M129L G207A D223G | 93 |
| T103G M129L G207A D223G | 90 |
| M83L T103G M129L D223G | 89 |
| T103G M129L G207A D223G P303K | 88 |
| S197G G207A D223G T244P A284N P303K V315I | 82 |
| T103G M129L D223G P303K | 77 |
| S31R M83L T103G M129L G207A D223G | 75 |
| M83L T103G M129L G207A D223G S250R | 72 |
| M83L T103G M129L G207A D223G G246A | 72 |
| M83L T103G G114A M129L G207A D223G | 70 |
| M83L T103G M129L G207A D223G A263P | 70 |
| S197G D223G T244P A284N P303K V315I | 67 |

### Example 3

### Oxidation Stability

Improved CALB variants in oxidation stability were found by measuring the residual activity after incubation with an oxidation agent.

The oxidation stability assay was performed by preparing a 0.5 mg/ml purified lipase solution in a 50 mM Tris-Acetate buffer pH 7.0 (Trizma base Sigma T6066) and acetic acid (Merck-60006325001730) containing 0% or 15% (V/V) hydrogen peroxide (Merck 61765305001730). Aliquots of 50 microliter of this enzyme solution were placed in PCR tubes (Axygen-PCR-08S-CPC) and incubated in a PCR machine (Applied Biosystem-Geneamp PCR System 9700) at a temperature of 40° C. for 20 minutes. The hydrogen peroxide treated samples were immediately stored at 4° C. until use.

For preparing the reaction mixture, the treated enzyme samples were diluted sixteen times using 50 mM hepes (Sigma H3375) buffer containing 10 mM Calcium chloride (Sigma C5080) and 0.4% Triton X-100 (Sigma T8787) pH 7.0. Aliquots of 20 microliter of diluted enzyme sample was transferred to a 96 well plate (Nunc-96F Micro well plate 269620) and to this 80 microliter of 50 mM hepes (Sigma H3375) buffer containing 10 mM Calcium chloride (Sigma C5080) and 0.4% Triton X-100 (Sigma T8787) pH 7.0 were added. A pNP Butyrate substrate solution was prepared by solubilizing 2 mM 4-nitrophenyl butyrate (Sigma N9876) in a 50 mM hepes (Sigma H3375) buffer containing 10 mM Calcium chloride (Sigma C5080) and 0.4% Triton X-100 (Sigma T8787) pH 7.0. Aliquots of 100 microliter substrate solution were added to the 100 microliter diluted enzyme solution.

The reaction mixtures were incubated at 25° C. and absorbances were measured at 405 nm for 5 minutes using a Microtiter Plate Reader (Spectra Max M5, Molecular Devices).

The residual activity percentage (RA %) is calculated as (the activity at 15% hydrogen peroxide divided by the activity at 0% hydrogen peroxide and multiplied by 100) %

TABLE 3
Oxidation stability. Residual activity after
20 min incubation at 15% hydrogen peroxide

| Variant | RA % |
|---|---|
| CalB wild type | 22 |
| M83L | 29 |
| G207A | 59 |
| T103G | 44 |
| M129L | 52 |
| A148P | 20 |
| D223G | 51 |
| S197G | 45 |
| P303K | 27 |
| G44A | 2 |
| G114A | 12 |
| T174N | 25 |
| G19A | 11 |
| G246A | 1 |
| S250R | 16 |
| M83L M129L G207A D223G | 89 |
| M83L T103G M129L A148P | 86 |
| M83L T103G M129L D223G | 85 |
| M83L T103G M129L G207A D223G | 85 |
| T103G M129L G207A D223G P303K | 84 |
| M83L T103G M129L | 84 |
| T103G M129L S197G G207A D223G | 83 |
| T103G M129L G207A D223G | 81 |
| G44A M83L T103G M129L G207A D223G | 80 |
| T103G M129L G207A P303K | 79 |
| T103G M129L | 76 |
| T103G A148P S197G G207A | 76 |
| M83L T103G G114A M129L G207A D223G | 75 |
| T103G M129L A148P | 75 |
| T103G M129L G207A | 74 |
| T103G M129L D223G | 73 |
| G19A M83L T103G M129L G207A D223G | 72 |
| M129L S197G G207A D223G P303K | 72 |
| T103G M129L T174N P303K | 71 |
| T103G M129L S197G G207A D223G P303K | 71 |
| M83L T103G M129L G207A D223G G246A | 68 |
| M83L T103G M129L G207A D223G S250R | 68 |
| M83L T103G | 68 |

### Example 4

### Methanol Stability

Improved CalB variants in methanol stability were found by measuring the residual activity after incubation with methanol.

The methanol stability assay was performed by preparing a 0.5 mg/ml purified lipase solution in a 50 mM Tris-Acetate buffer pH 7.0 (Trizma base Sigma T6066) and acetic acid (Merck-60006325001730) containing 0% or 50% (V/V) absolute methanol HPLC grade (Fisher Scientific 43607). Aliquots of 50 microliter of this enzyme solution were placed in PCR tubes (Axygen-PCR-08S-CPC) and incubated in a PCR machine (Applied Biosystem-Geneamp PCR System 9700) at temperature of 47° C. for 20 minutes. The methanol treated samples were immediately stored at 4° C. until use.

For preparing the reaction mixture, the treated enzyme samples were diluted sixteen times using 50 mM hepes (Sigma H3375) buffer containing 10 mM Calcium chloride (Sigma C5080) and 0.4% Triton X-100 (Sigma T8787) pH 7.0. Aliquots of 20 microliter of diluted enzyme sample was transferred to a 96 well plate (Nunc-96F Micro well plate 269620) and to this 80 microliter of 50 mM hepes (Sigma H3375) buffer containing 10 mM Calcium chloride (Sigma C5080) and 0.4% Triton X-100 (Sigma T8787) pH 7.0 were added. A pNP Butyrate substrate solution was prepared by solubilizing 2 mM 4-nitrophenyl butyrate (Sigma N9876) in a 50 mM hepes (Sigma H3375) buffer containing 10 mM Calcium chloride (Sigma C5080) and 0.4% Triton X-100 (Sigma T8787) pH 7.0. Aliquots of 100 microliter substrate solution were added to the 100 microliter diluted enzyme solution.

The reaction mixtures were incubated at 25° C. and absorbances were measured at 405 nm for 5 minutes using a Microtiter Plate Reader (Spectra Max M5, Molecular Devices).

The residual activity percentage (RA %) is calculated as (the activity at 50% methanol divided by the activity at 0% methanol and multiplied by 100) %.

TABLE 4
Methanol stability of CALB variants. Residual activity
(RA) after 20 min incubation in 50% methanol.

| Variant | RA % |
|---|---|
| CalB wild type | 55 |
| G41A | 55 |
| T103G | 61 |
| M83L | 67 |
| M129L | 46 |
| A148P | 67 |
| S197G | 62 |
| G207A | 75 |
| P303K | 45 |
| G44A | 42 |
| T174N | 46 |
| S31R | 59 |
| T244P | 95 |
| G114A | 51 |
| A284N | 88 |
| V315I | 62 |
| D223G | 58 |
| A263P | 39 |
| S250R | 48 |
| G41A M129L A148P S197G G207A D223G | 100 |
| T103G M129L G207A D223G | 99 |
| M83L T103G M129L G207A D223G A263P | 99 |
| M83L T103G M129L D223G | 98 |
| T103G A148P S197G G207A | 98 |
| M83L T103G M129L G207A D223G S250R | 97 |
| T103G M129L G207A | 97 |
| T103G M129L G207A P303K | 96 |
| G44A M83L T103G M129L G207A D223G | 96 |
| T103G M129L T174N P303K | 96 |
| S31R M83L T103G M129L G207A D223G | 95 |
| S197G G207A D223G T244P A284N P303K V315I | 95 |
| T103G M129L G207A D223G P303K | 95 |
| T244P | 95 |
| M83L T103G G114A M129L G207A D223G | 95 |

### Example 5

### Transesterification Activity

The immobilization media (Lewatit VP OC 1600) was washed with ethanol and repeatedly washed with reverse osmosis water. The immobilization media was then dried in a decicator to remove moisture. 20 mg of enzyme [in liquid form; HEPES (50 mM), pH 7] was added per gram of the immobilizing media and the suspension was gently shaken for 18 hours at 4° C. The suspension was allowed to settle down; the supernatant was decanted and run on a 12% SDS-PAGE to ascertain that the extent of immobilization. The immobilization media was dried in a decicator to remove moisture.

20 mg of immobilized enzyme sample was placed in a 2 ml micro centrifuge tube and to this 900 microliter of peanut oil and 100 microliter of ethanol are added and incubated at 30° C. for 24 hr at 750 rpm. After incubation, the oil was separated from the immobilization media, transferred to a fresh micro centrifuge tube and dried at 60° C. for 5 hour to remove ethanol and water. This sample was used for GC analysis.

50 microliter sample was transferred to a GC vials, 50 microliter of 20 mg/ml Internal standard (methyl hepta deconoate) was added and the volume was made up to 1000 microliter in n-hexane (HPLC grade). 1 microliter of this sample was injected to a GC fitted with a DB-5 column (Agilent) and using a split ratio of 1:50. The injector temperature was 250° C. and detector temperature 280° C. The initial temperature was set at 90° C. and increased to 150° C. with 20° C./min and from 150° C. to 250° C. with 5° C./min increment and final temperature raised to 300° C. with 20° C./min increment.

TABLE 5
Transesterification assay. Fatty acid ethyl
esters in percentage of theoretical yield.

| Variant | % FAEE |
|---|---|
| CalB wild type | 1 |
| G41A | 25 |
| T103S | 8 |
| M129L D223G P303K | 30 |
| T103G M129L P303K G207A D223G | 28 |
| M129L G207A D223G P303K | 28 |
| T103G M129L D223G | 26 |
| T103G M129L D223G G207A S197G | 23 |
| T103G M129L D223G P303K | 23 |
| T103G M129L D223G G207A M83L | 23 |
| T103G M129L I255S | 13 |
| T103G M129L N96E | 13 |
| T103G M129L G254S | 13 |
| M83L T103G M129L D223G | 12 |
| T103G M129L A251P | 12 |
| T103G M129L N97Q | 8 |
| T103G M129L G207A P303K | 6 |
| T103G M129L P303K | 6 |
| T103G M129L G207A | 6 |
| T103G M129L N97T | 6 |
| T103G M129L | 5 |
| T103G M129L P303K T174N | 5 |
| M83L T103G M129L | 5 |
| L278A | 4 |
| M83L T103G M129L A148P | 3 |
| T103G A148P G207A S197G | 3 |
| T103G M129L S197G G207A D223G G41A | 3 |

### Example 6

### Specific Lipase Activity (LU)

The specific lipase activity was determined in LU/mg enzyme protein.

TABLE 6
Lipase activity, specific activity (LU/mg)

| Variant | LU/mg |
|---|---|
| CalB wild type | 663 |
| G41S | 817 |
| M83I | 815 |
| L20I | 797 |
| V215I | 776 |
| A8P | 764 |
| A251S | 750 |
| G41A | 1376 |
| L278A | 3293 |
| A284N | 1047 |
| M129L S197G G207A D223G P303K | 2125 |
| S197G A284N P303K | 1235 |
| M83L P303K | 1164 |
| S197G T244P A284N V315I | 1149 |
| M129L G207A D223G | 1059 |
| M129L D223G | 852 |

1-17. (canceled)

18. A method of preparing a variant of a parent polypeptide comprising:
(a) providing an amino acid sequence of a parent polypeptide;
(b) substituting at least one amino acid residue at a position in the sequence corresponding to any of positions: 41, 83, 129, 207 or 284 or 284 in SEQ ID No: 2;
(c) selecting a variant with lipolytic activity, which compared to the parent polypeptide has an improved property, and has an amino acid sequence with at least 60% identity to the mature polypeptide of SEQ ID No: 2; and
(d) recovering the variant.

19. The method of claim 18, wherein the parent polypeptide consists of or comprises an amino acid sequence with at least 60% identity to a lipase selected from the group consisting of: Candida Antarctica lipase B (SEQ ID No: 2), Hyphozyma sp. lipase (SEQ ID No: 3), Ustilago maydis lipase (SEQ ID No: 4), Giberella zeae lipase (Fusarium graminearum lipase, SEQ ID No: 5), Debaryomyces hansenii lipase (SEQ ID No: 6), Aspergillus fumigates lipase (SEQ ID No: 7), Aspergillus oryzae lipase (SEQ ID No: 8), and Neurospora crassa lipase (SEQ ID No: 9).

20. The method of claim 18, further comprising substituting at least one further position in the amino acid sequence of the parent polypeptide corresponding to any of positions 103, 197, 223 or 278 in SEQ ID No: 2.

21. An isolated variant of a parent polypeptide, wherein said variant is:
(a) a polypeptide comprising a substitution of at least one amino acid residue at a position corresponding to any of positions: 41, 83, 129, 207 or 284 of SEQ ID No: 2, wherein said variant has lipolytic activity, which variant compared to the parent polypeptide has improved stability, and has an amino acid sequence with at least 60% identity to the mature polypeptide of SEQ ID NO: 2;
(b) a polypeptide encoded by a polynucleotide that hybridizes under at least low stringency conditions with (i) the mature polypeptide coding sequence of SEQ ID No: 1, (ii) the genomic DNA sequence comprising the mature polypeptide coding sequence of SEQ ID NO: 1, or (iii) a full-length complementary strand of (i) or (ii); or
(c) a polypeptide encoded by a polynucleotide comprising a nucleotide sequence having at least 60% identity with the mature polypeptide coding sequence of SEQ ID NO: 1.

22. The variant of claim 21, wherein the parent polypeptide consists of or comprises an amino acid sequence with at least 60% identity to is a lipase selected from the group consisting of: Candida Antarctica lipase B (SEQ ID No: 2), Hyphozyma sp. lipase (SEQ ID No: 3), Ustilago maydis lipase (SEQ ID No: 4), Giberella zeae lipase (Fusarium graminearum lipase, SEQ ID No: 5), Debaryomyces hansenii lipase (SEQ ID No: 6), Aspergillus fumigates lipase (SEQ ID No: 7), Aspergillus oryzae lipase (SEQ ID No: 8), and Neurospora crassa lipase (SEQ ID No: 9).

23. The variant of claim 21, having at least 80% identity to the mature polypeptide of SEQ ID NO: 2.

24. The variant of claim 21, having at least 90% identity to the mature polypeptide of SEQ ID NO: 2.

25. The variant of claim 21, having at least 95% identity to the mature polypeptide of SEQ ID NO: 2.

26. The variant of claim 21, further comprising substitution of the amino acid sequence of the parent polypeptide corresponding to any of positions 103, 197, 223 or 278 in SEQ ID NO: 2.

27. The variant of claim 26, wherein the substitutions 41A, 83L, 103G, 129L, 197G, 207A, 223G or 278A.

28. The variant of claim 21, wherein the substitutions are selected from the group consisting of:
(a) G41A
(b) M83L
(c) M83L+T103G
(d) M83L+T103G+M129L
(e) M83L+T103G+M129L+G207A+D223G
(f) M83L+T103G+M129L+D223G
(g) T103G
(h) T103G+M129L
(i) T103G+M129L+S197G+G207A+D223G
(j) T103G+M129L+G207A
(k) T103G+M129L+G207A+D223G
(l) T103G+M129L+D223G
(m) M129L
(n) S197G
(o) G207A
(p) D223G
(q) M83L+T103G+M129L+A148P
(r) N97Q+T103G+M129L
(s) T103G+W104H+ M129L
(t) T103G+M129L+A148P
(u) T103G+M129L+S197G+G207A+D223G+P303K
(v) T103G+M129L+G207A+P303K
(w) T103G+M129L+D223G+P303K
(x) T103G+M129L+P303K
(y) T103G+P303K,
(z) P303K+S197G+A284N+D223G+T244P+V3151
(aa) M129L+S197G+G207A+D223G+G41A+A148P
(bb) T103G+M129L+S197G+G207A+D223G+G41A+
(cc) T103G+M129L+S197G+G207A+D223G+G41A+A148P
(dd) M129L+D223G+P303K
(ee) M129L+G207A+D223G+P303K
(ff) S197G+A284N+P303K
(gg) S197G+T244P+A284N+V3151
(hh) T103G+A148P+G207A
(ii) T103G+A148P+G207A+S197G
(jj) T103G+M129L
(kk) T103G+M129L+A251P
(ll) T103G+M129L+D223G
(mm) T103G+M129L+D223G+G207A
(nn) T103G+M129L+D223G+G207A+M83L
(oo) T103G+M129L+D223G+G207A+M83L+A263P
(pp) T103G+M129L+D223G+G207A+M83L+D134P
(qq) T103G+M129L+D223G+G207A+M83L+G114A
(rr) T103G+M129L+D223G+G207A+M83L+G19A
(ss) T103G+M129L+D223G+G207A+M83L+G226A
(tt) T103G+M129L+D223G+G207A+M83L+G246A
(uu) T103G+M129L+D223G+G207A+M83L+G288P
(vv) T103G+M129L+D223G+G207A+M83L+G41A+L278A
(ww) T103G+M129L+D223G+G207A+M83L+G41S
(xx) T103G+M129L+D223G+G207A+M83L+G44A
(yy) T103G+M129L+D223G+G207A+M83L+N264P
(zz) T103G+M129L+D223G+G207A+M83L+S250R
(aaa) T103G+M129L+D223G+G207A+M83L+S31R
(bbb) T103G+M129L+D223G+G207A+S197G
(ccc) T103G+M129L+D223G+G207A+S197G+P303K
(ddd) T103G+M129L+D223G+P303K
(eee) T103G+M129L+G254S
(fff) T103G+M129L+G254T
(ggg) T103G+M129L+1255S
(hhh) T103G+M129L+1255T
(iii) T103G+M129L+N96E
(jjj) T103G+M129L+N97Q
(kkk) T103G+M129L+N97T
(lll) T103G+M129L+P303K+G207A+D223G
(mmm) T103G+M129L+P303K+T174N.

29. An isolated polynucleotide encoding the variant of claim 21.

30. A nucleic acid construct comprising the isolated polynucleotide of claim 29.

31. An expression vector comprising the nucleic acid construct of claim 30.

32. A host cell comprising the nucleic acid construct of claim 30.

33. A composition comprising the variant of claim 21.
